(19) United States
(12) Patent Application Publication
(10) Pub. No.: US 2007/0207103 A1
(43) Pub. Date: Sep. 6, 2007

(54) Lipophilic personal care composition

(75) Inventor: Hisatoshi Masuda, Moriyama (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/714,426

(22) Filed: Mar. 6, 2007

Related U.S. Application Data
(60) Provisional application No. 60/779,545, filed on Mar. 6, 2006.

(51) Int. Cl. A61K 8/49; A61Q 1/06

(52) U.S. Cl. 424/64; 514/352; 514/460; 514/506; 514/772; 514/788

(57) ABSTRACT

The present invention is directed to a water-in-oil emulsion composition comprising: a lipophilic personal care composition comprising by weight: (a) from about 1% to about 30% of a polyglycerin fatty acid ester having a polyglycerin of 2-20 units and at least one branched fatty acid residue of 8 to 22 carbons; (b) from about 0.01% to about 6% of glycerin, wherein the amount of glycerin is no more than 20% of the polyglycerin fatty acid ester; (c) from about 0.001% to no more than an amount dissolvable in glycerin of a water-soluble skin benefit agent; and (d) a lipophilic carrier which is substantially free of surfactant.

### CROSS REFERENCE TO RELATED APPLICATION

This application claims the benefit of U.S. Provisional Application No. 60/779,545 filed on Mar. 6, 2006.

### FIELD OF THE INVENTION

The present invention relates to lipophilic personal care compositions comprising a polyglycerin fatty acid ester, glycerin, and a water-soluble skin benefit agent, wherein the water-soluble skin benefit agent is incorporated in the composition in a stable manner. The composition is particularly useful for making cosmetic compositions such as lipstick, lipgloss, foundations, and creams.

### BACKGROUND

Recently, the lipstick market has been segmented into those consumers who seek wear benefit, moisturizing benefit, and balance of wear and moisturizing benefits. Lipsticks are primarily made of lipophilic or hydrophobic materials. Lipsticks designed for providing a moisturizing benefit to the lips have a tendency of further containing polar solvents, or other moisturizing components which are more or less hydrophilic. The use of association structures and gel structures has been suggested to bind such polar solvents in the lipophilic matrix of the lipstick, such as in PCT publication WO 02/26198. While such lipstick compositions provide a favorable moisturizing benefit to the lips, some compositions were unsatisfactory in physical stability, and some others required uncommon equipment or extra processing steps.

The lips are exposed to damage by the environment all year round. Such damage is noticed by the consumer via dryness and tight feeling of the lips. The desire to have moisturizing, as well as soft, elastic texture of lips having plumpy look remains an unmet need. Further, there is yet an unmet need to improve the dullness of lip color according to age. Meanwhile, certain skin benefit agents which provide such function to the lips are water-soluble. It is generally known to the artisan that incorporation of water-soluble ingredients in a lipophilic composition in a stable manner requires usage of a surfactant. Usage of surfactant, however, is undesired from a performance point of view, as surfactants are known as skin irritants.

Cosmetic compositions containing polyglycerin fatty acid ester are disclosed in Japanese patent publications A-2005-132729, A-2005-132730, A-2005-239592, and A-2005-247739. Lipophilic cosmetic compositions containing polyglycerin fatty acid ester are disclosed in Japanese patent publications A-2005-239590, and A-2005-239591. Lipophilic personal care compositions which can further accommodate water-soluble skin benefit agents in a stable manner is desired.

Based on the foregoing, there is a need for a lipophilic personal care composition which provides improved moisturizing and improved texture of the skin, while also having good physical stability. There is further a need for a lipophilic personal care composition which improves dullness of skin color. There is also a need for a personal care composition which can be manufactured by utilizing commonly used equipment.

### SUMMARY

The present invention is directed to a lipophilic personal care composition comprising by weight:
(a) from about 1% to about 30% of a polyglycerin fatty acid ester having a polyglycerin of 2-20 units and at least one branched fatty acid residue of 8 to 22 carbons;
(b) from about 0.01% to about 6% of glycerin, wherein the amount of glycerin is no more than 20% of the polyglycerin fatty acid ester;
(c) from about 0.001% to no more than an amount dissolvable in glycerin of a water-soluble skin benefit agent; and
(d) a lipophilic carrier which is substantially free of surfactant. The present invention is also directed to the lipophilic personal care composition described above wherein the composition is prepared by the steps of:
(1) dissolving the water-soluble skin benefit agent into glycerin;
(2) dissolving the product of step (1) into the polyglycerin fatty acid ester; and
(3) mixing the product of step (2) with the anhydrous lipophilic carrier.

The composition of present invention satisfies the need for improved moisturization and improved texture of the skin, while also having good physical stability.

These and other features, aspects, and advantages of the present invention will become evident to those skilled in the art from a reading of the present disclosure with the appended claims.

### DETAILED DESCRIPTION

The following is a list of definitions for terms used herein.

“Comprising” means that other steps and other ingredients which do not affect the end result can be added. This term encompasses the terms “consisting of” and “consisting essentially of”.

All percentages are by weight of total composition unless specifically stated otherwise.

All cited references are incorporated herein by reference in their entireties. Citation of any reference is not an admission regarding any determination as to its availability as prior art to the claimed invention.

All ratios are weight ratios unless specifically stated otherwise.

The present invention, in its product and process aspects, is described in detail as follows.

### Polyglycerin Fatty Acid Ester

The composition of the present invention comprises a polyglycerin fatty acid ester having a polyglycerin of 2-15 units and at least 5 fatty acid residues attached thereto, wherein the fatty acid residue has 18 to 24 carbons. The polyglycerin fatty acid ester useful herein is liquid at room temperature, has high water holding capability, and is compatible with many oily materials useful for making lipophilic personal care compositions. It has been surprisingly found that, the polyglycerin fatty acid ester is capable of dissolving up to 20% of its weight of glycerin. By first dissolving the water-soluble skin benefit agent into glycerin, the glycerin solution can thus be dissolved into the polyglycerin fatty acid ester herein, and incorporated in the present lipophilic composition in a stable manner.

The polyglycerin fatty acid ester is comprised by weight of the entire composition at from about 1% to about 30%. In the particularly preferred lipstick embodiment, the polyglycerin fatty acid ester is comprised by weight of the entire composition at from about 5% to about 30%, preferably from about 10% to about 25%.

Polyglycerin fatty acid esters useful herein include esters made of polyglyceryl-2, polyglyceryl-4, polyglyceryl-6, or polyglyceryl-10; with at least 5 isostearate residues.

Commercially available polyglycerin fatty acid esters useful herein include polyglyceryl-10 nonaisostearate with tradename S-FACE IS-1009P available from Sakamoto Pharmaceuticals.

### Glycerin

The composition of the present invention comprises from about 0.01% to about 6%, preferably from about 1% to about 6% by weight of the entire composition of glycerin, wherein the amount of glycerin is no more than 20% of the polyglycerin fatty acid ester. Glycerin is used as a solvent for dissolving the water-soluble skin benefit agents which otherwise would not be compatible in the lipophilic carrier base. Glycerin itself also provides moisturizing benefit.

Glycerin is commercially available, for example, from Asahi Denka.

### Water-Soluble Skin Benefit Agent

The composition of the present invention comprises a cosmetically acceptable water-soluble skin benefit agent. The water-soluble skin benefit agent herein can be liquid or solid, and is incorporated in an amount of from about 0.01% to an amount dissolvable in glycerin, preferably from about 0.01% to about 1% of the entire composition. By first dissolving the water-soluble skin benefit agent into glycerin, the glycerin solution can thus be dissolved into the polyglycerin fatty acid ester herein, and incorporated in the present lipophilic composition in a stable manner.

Suitable water-soluble skin benefit agents herein include flavonoid compounds, vitamin B3 compounds, ascorbic acid compounds, other skin benefit agents, and mixtures thereof.

Water-soluble flavonoid compounds are useful as water-soluble skin benefit agents herein. Generally, flavonoid compounds are known to be effective in strengthening collagen structure. Without being bound by theory, it is believed that the water-soluble flavonoid compounds herein are believed to enhance blood circulation, and therefore enhance skin lightness. In the preferred lipstick embodiment, it is believed that water-soluble flavonoid compounds are particularly preferred for improving dullness of skin color.

Water-soluble flavonoid compounds useful herein are selected from the group consisting of substituted flavanones, substituted flavones, substituted chalcones, substituted isoflavones, and mixtures thereof. By the term “substituted” as used herein means flavonoid compounds wherein one or more hydrogen atoms of the skeleton structure as described above has been independently replaced with hydroxyl, C1-C8 alkyl, C1-C4 alkoxyl, O-glycoside, and the like or a mixture of these substituents. The water-soluble flavonoid compounds useful herein can be synthetic materials derived or modified from naturally sourced material. Preferred water-soluble flavonoid compounds are glycosylated, alkylated or acylated from naturally sourced material.

A particularly useful group of glycoside flavonoids herein are those selected from the general structural formula (III)

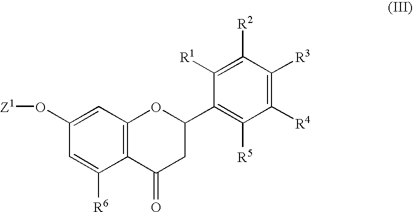

wherein R¹—R⁶ are independently selected from the group consisting of H, OH, alkoxy and hydroxyalkoxy, wherein the alkoxy or hydroxyalkoxy groups are branched or unbranched and have 1-18 carbon atoms, and wherein Z¹ is selected from the group consisting of mono- and oligoglycoside radicals. Z¹ is preferably selected from the group consisting of hexosyl radicals, more preferably rhamnosyl radicals and glucosyl radicals. It is also advantageous to use other hexosyl radicals, for example allosyl, altrosyl, galactosyl, gulosyl, idosyl, mannosyl and talosyl. It may also be advantageous according to the invention to use pentosyl radicals.

Another particularly useful group of glycoside flavonoids herein are those selected from the general structural formula (IV)

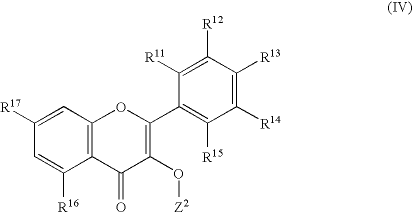

wherein R′¹-R¹⁷ are independently selected from the group consisting of H, OH, alkoxy and hydroxyalkoxy, wherein the alkoxy or hydroxyalkoxy groups are branched or unbranched and have 1-18 carbon atoms, and wherein Z² is selected from the group consisting of mono- and oligoglycoside radicals. Z² is preferably selected from the group consisting of hexosyl radicals, more preferably rhamnosyl radicals and glucosyl radicals. It is also advantageous to use other hexosyl radicals, for example allosyl, altrosyl, galactosyl, gulosyl, idosyl, mannosyl and talosyl. It may also be advantageous according to the invention to use pentosyl radicals.

In one particularly preferred embodiment of the present invention, the glycoside flavonoid is selected from the group consisting of hesperidin, glucosyl hesperidin, rutin, glucosyl rutin, glucosyl myricitrin, glucosyl isoquercitrin, glucosyl quercitrin, methyl hesperidin, and mixtures thereof. These glucoside flavonoid compounds can be obtained by bio-chemical methods from related natural flavonoid compounds. The glucosyl group(s) can be connected to one or more hydroxides of the original substances.

A representative formula of glucosyl rutin is as follows:

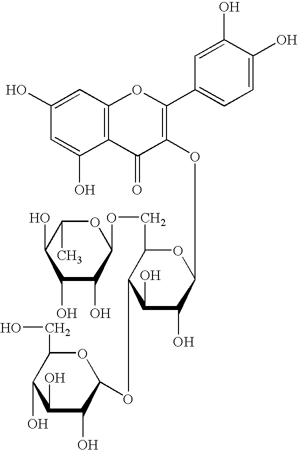

A representative formula of glucosyl hesperidin is as follows:

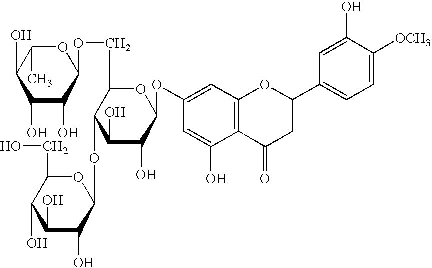

Another useful alkylated flavonoid compound, methyl hesperidin, has the general structural formula (V)

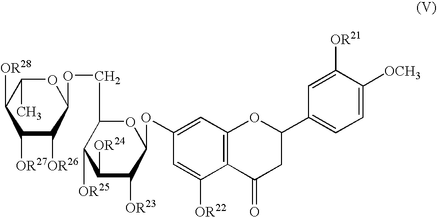

wherein R²¹—R²⁸ are independently selected from the group consisting of H or methyl, wherein at least one of R²¹-R²⁸ is methyl.

Commercially available water-soluble flavonoid compounds include methylhesperidin available from Alps Pharmaceutical Industry Co. Ltd. (Japan); and glucosyl hesperidin and glucosyl rutin available from Hayashibara Biochemical Laboratories, Inc. (Japan) and Toyo Sugar Refining Co. Ltd. (Japan).

Vitamin B3 compounds are useful as water-soluble skin benefit agents herein. Generally, vitamin B3 compounds are known to provide, by itself, a precursor for nicotinamide adenine dinucleotide phosphate (NADP) family and its reduced form (NADPH) family of coenzymes, which enhance many metabolic enzyme reactions on the skin. Vitamin B3 compounds are also known to provide reduction in trans-epidermal water loss and excess dermal glycosaminoglycans, which are indicators for skin barrier properties. Vitamin B3 compounds useful herein include, for example, those having the formula:

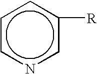

wherein R is —CONH₂ (e.g., niacinamide) or —CH₂OH (e.g., nicotinyl alcohol); derivatives thereof; and salts thereof. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form. Commercially available water-soluble vitamin B₃ compounds that are highly useful herein include niacinamide USP available from Reilly.

Ascorbic acid compounds are useful as water-soluble skin benefit agents herein include ascorbic acid compounds. Ascorbic acid compounds are known to provide skin lightening benefit. Ascorbic acid compounds useful herein include, ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein includes, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available water-soluble ascorbic compounds include magnesium ascorbyl phosphate available from Showa Denko, 2-o-D-glucopyranosyl-L-ascorbic acid available from Hayashibara and sodium L-ascorbyl phosphate with tradename STAY C available from Roche.

Other water-soluble skin benefit agents useful herein include panthenol, bacterial cultured mediums, allantoin, sodium lactate, PCA soda, amino acids, urea, sodium hyaluronate, chondroitin sulfate, collagen, elastin, pectin, carageenan, sodium alginate, trehalose, tuberose saccharide, chitin derivatives, chitosan derivatives, water-soluble plant extracts, and mixtures thereof. Commercially available water-soluble skin benefit agents include sodium hyaluronate with tradenames ACTIMOIST available from Active Organics, AVIAN SODIUM HYALURONATE series available from Intergen, and HYALURONIC ACID Na available from Ichimaru Pharcos.

### Lipophilic Composition

The composition of the present invention is made of a single continuous lipophilic phase that is substantially free of surfactant. By substantially free of surfactant, what is meant is that there is no surfactant intentionally included. Surfactants herein include emulsifiers. By providing the composition substantially free of surfactants, the composition has reduced risk of causing irritation to the skin or scalp. The present composition may be anhydrous. While there could be a small amount of water included in the composition, such water is within the amount that could be completely solubilized in the lipophilic components of the composition. For example, the polyglycerin fatty acid ester has a certain water holding capability. The present invention excludes compositions that are emulsions.

The lipophilic personal care composition of the present invention may take various consumer product forms, such as lipstick, lipbalm, lipgloss, foundation, skin cream, antiperspirant, hair treatment cream, ointment, and others. The present composition is particularly useful for making cosmetic compositions such as lipstick, lipgloss, foundations, and creams.

For providing a stable composition, the composition of the present invention is prepared by the steps of:
(1) dissolving the water-soluble skin benefit agent into glycerin;
(2) dissolving the product of step (1) into the polyglycerin fatty acid ester; and
(3) mixing the product of step (2) with the anhydrous lipophilic carrier.

The lipophilic carrier of the present intention that is substantially free of surfactants may contain various lipophilic components for making the remainder of the composition. Such components can be any that are useful for personal care products, and which characterize the product. Nonlimiting examples of the other lipophilic components include N-long chain acyl acidic amino acid ester of sterol, dimer acid esters, and other nonvolatile oils. For providing the preferred cosmetic compositions of the present invention, components such as solid wax, color powders, and other components may be included. One highly preferred embodiment of the present invention is lipstick. The present lipstick composition comprises solid wax for solidifying the composition, and color powders for providing color to the lips.

### N-Long Chain Acyl Acidic Amino Acid Ester or Diester of Sterol

The composition of the present invention comprises a N-long chain acyl acidic amino acid ester of sterol having formula (I):

XOC(O)—(CH2)n-CH(NHCOR)—C(O)OY (I)

wherein one of X and Y is an ester residue of sterol selected from the group consisting of phytosterol, cholesterol, lanosterol, stigniasterol, its hydrogenates, and its derivatives, and the other is selected from H, an alkyl or alkenyl of 8-30 carbons, and ester residue of monohydric solid alcohol of 12-38 carbons; COR is a long chain acyl group of 8-22 carbons; and n is an integer of 1 or 2. The ester may be a monoester or diester. Preferably, the one of X and Y which is not the ester residue of sterol is selected from 2-octyldodecyl alcohol, isostearyl alcohol, oleyl alcohol, cetyl alcohol, benenyl alcohol, and mixtures thereof. The N-long chain acyl acidic amino acid ester of sterol of the present invention has a water holding capability of at least 400%, and is liquid at 25° C. By having such physical properties, it is believed that the N-long chain acyl acidic amino acid ester of sterol herein is effective in alleviating damaged skin, compatible with many oily materials useful for making lipophilic personal care compositions, and provide a favorable feel to the skin.

The N-long chain acyl acidic amino acid ester of sterol is comprised by weight of the entire composition at from about 5% to about 50%. In the particularly preferred lipstick embodiment, the N-long chain acyl acidic amino acid ester of sterol is comprised by weight of the entire composition at from about 5% to about 25%, preferably from about 10% to about 20%.

Commercially available N-long chain acyl acidic amino acid ester of sterol useful herein include: phytosteryl/octyldodecyl lauroyl glutamate with tradename ELDEW PS-203, and cholesteryl/octyldodecyl lauroyl glutamate with tradename ELDEW CL-202, both available from Ajinomoto Ltd.

### Dimer Acid Ester

The composition of the present invention comprises a dimer acid ester which may be a monoester or diester of dimer acid, wherein at least one group is esterified with a sterol selected from the group consisting of phytosterol, cholesterol, lanosterol, stigrnasterol, its hydrogenates, and its derivatives. The dimer acid ester may also be a diester of dimer acid esterified with fatty alcohol. The remaining group or both groups may be esterified with a straight chain alcohol having 1-34 carbons, a branched chain alcohol having 3-34 carbons, or an unsaturated alcohol having 6-34 carbons. The dimer acid ester of the present invention preferably has a water holding capability of at least 240%, and is liquid at 25° C.

The dimer acid ester is comprised by weight of the entire composition at from about 5% to about 50%. In the particularly preferred lipstick embodiment, the dimer acid ester may be comprised by weight of the entire composition at from about 5% to about 25%, preferably from about 10% to about 20%.

Commercially available dimer acid esters include: phytosteryl isostearyl dimer dilinoleate with tradename LUSPLAN PI-DA, dimer dilinoleyl hydrogenated rosinate with tradename LUSPLAN DD-DHR, and dimer dilinoleyl dimer dilinoleate: with tradenames LUSPLAN DD-DA5 and LUSPLAN DD-DA7, all available from Nippon Fine Chemical Co., Ltd.

### Other Non-Volatile Oil

The composition of the present invention may further comprise other non-volatile oils suitable for personal use. The non-volatile oil may be comprised by weight of the entire composition at from about 1% to about 50%, preferably from about 5% to about 30%.

Non-volatile oils useful herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, diglyceryl sebacate, neopentyl glycol di(2-ethyl hexanoate), diisopropyl dimerate, tocopherol, tocopherol acetate, castor oil, turtle oil, mink oil, eggyolk oil, glycerol trioctanate, glycerol triisopalmitate, polyglyceryl-2 triisostearate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, shea butter, and mixtures thereof. Commercially available oils include, for example, tridecyl isononanoate with tradename Crodamol TN available from Croda, Hexalan available from Nisshin Oil Mills, Ltd., tocopherol acetates available from Eisai, polyglyceryl-2 triisostearate with tradename COSMOL 43V available from Nisshin Oil Mills, Ltd., and shea butter with tradename CROPURE SB available from Croda.

Non-volatile oils useful herein also include polyalkyl or polyaryl siloxanes with the following structure (II)

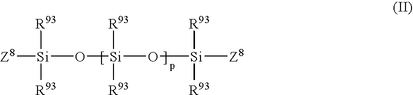

wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known by CTFA name dimethicone, is especially preferred. These silicone compounds are available, for example, from the General Electric Company in their Viscasil® and SF 96 series, and from Dow Corning in their Dow Corning 200 series.

Polyalkylaryl siloxane fluids can also be used and include, for example, polymethylphenylsiloxanes. These siloxanes also known by CTFA name phenyl trimethicone are available, for example, from the General Electric Company as SF 1075 methyl phenyl fluid, from Dow Corning as 556 Cosmetic Grade Fluid, or from ShinEtsu Silicone as Silicone Oil KF-56.

Non-volatile oils also useful herein are the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

Non-volatile oils also useful herein are low viscosity vegetable oils having a viscosity of no more than about 300 mPas. Non-limited examples of low viscosity vegetable oils useful herein are: sweet almond oil, rapeseed oil, fennel oil, olive oil, orange oil, canola oil, matricaria flower oil, cucumber oil, aleurites moluccana seed oil, cinnamon oil, corn oil, arctium lappa seed oil, sesame seed oil, rice bran oil, rice germ oil, camellia kissi seed oil, safflower seed oil, shea butter oil, job's tears seed oil, peppermint oil, soybean oil, tea seed oil, camellia japonica seed oil, rosa canina fruit oil, apricot kernel oil, palm kernel oil, palm oil, hybrid safflower seed oil, hybrid sunflower oil, peanut oil, sunflower seed oil, grape seed oil, jojoba oil, macadamia integrifolia nut oil, meadowfoam seed oil, coconut oil, eucalyptus oil, eucalyptus clobulus leaf oil, rosemary leaf oil, evening primrose oil, its hydrogenated products, and mixtures thereof. Preferred for use herein are macadamia integrifolia nut oil, meadowfoam seed oil, olive oil, rosa canina fruit oil, sunflower seed oil, coconut oil, sweet almond oil, shea butter oil, and mixtures thereof. Commercially available low viscosity vegetable oil include macadamia integrifolia nut oil with tradename FLORAMAC HAWAIIAN MACADAMIA available from Floratech, meadowfoam seed oil with tradename CROPURE MDF available from Croda, olive oil with tradename CROPURE OL available from Croda, jojoba oil with tradename Jojoba Oil available from Mitsuba Trading, and safflower seed oil with tradename Safflower Seed Oil available from NOF Corporation.

### Solid Wax

The composition of the present invention may further comprise a solid wax. For providing solid personal care compositions such as lipstick, solid foundations and antiperspirant sticks, solid wax is typically included. The amount of the solid wax is controlled to provide the desired hardness and strength to the product. In the particularly preferred lipstick embodiment, the solid wax is comprised by weight of the entire composition at from about 5% to about 20%, preferably from about 8% to about 15%.

The solid waxes useful herein are paraffin wax, microcrystalline wax, ozokerite wax, ceresin wax, camauba wax, candelilla wax, eicosanyl behenate, polyethylene wax, and mixtures thereof. A mixture of waxes is preferably used.

Commercially available solid waxes useful herein include: Candelilla wax NC-1630 available from Cerarica Noda Co., Ltd., Ozokerite Wax SP-1021 available from Strahl & Pitsch, eicosanyl behenate available from Cas Chemical, microcrystalline wax with tradename MULTIWAX 180-M Yellow available from Witco Chemical, and polyethylene wax with tradename PERFORMALENE PL available from New Phase Technology.

### Color Powders

The composition of the present invention may further comprise color powders. Herein, color powders include those which are white or nearly transparent. For providing the preferred cosmetic compositions, the color powder component is comprised by weight of the entire composition at from about 1% to about 50%, preferably from about 2% to about 45%. The amount and type of powders are selected depending on the desired characteristic of the product, for example, shade, coverage, UV protection benefit, and various skin feel.

The materials useful herein are clay mineral powders such as talc, mica, sericite, silica, magnesium silicate, synthetic fluorphlogopite, calcium silicate, aluminum silicate, bentonite and montomorillonite; pearl pigments such as alumina, barium sulfate, calcium secondary phosphate, calcium carbonate, titanium oxide, finely divided titanium oxide, zirconium oxide, zinc oxide, hydroxy apatite, iron oxide, iron titate, ultramarine blue, Prussian blue, chromium oxide, chromium hydroxide, cobalt oxide, cobalt titanate, titanium oxide coated mica; organic powders such as polyester, polyethylene, polystyrene, methyl metharylate resin, cellulose, 12-nylon, 6-nylon, styrene-acrylic acid copolymers, poly proprylene, vinyl chloride polymer, tetrafluoroethylene polymer, boron nitride, fish scale guanine, laked tar color dyes, and laked natural color dyes.

A certain percentage of spherical powders can be used. In the preferred foundation embodiments, the materials may be selected depending on the oil absorbing capability of the powders.

Hydrophobically treated powders can also be used. Such hydrophobically treated powders are made by treating the base material, as above, with a hydrophobical treatment agent, including: silicone such as Methicone, Dimethicone and perfluoroalkylsilane; fatty material such as stearic acid; metal soap such as aluminium dimyristate; aluminium hydrogenated tallow glutamate, hydrogenated lecithin, lauroyl lysine, aluminium salt of perfluoroalkyl phosphate, and mixtures thereof.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such additional components generally are used individually at levels of no more than about 5% by weight of the composition.

The composition of the present invention may further contain a nonvolatile dispersed silicone usually referred to as silicone gum. The term “silicone gum”, as used herein, means a polyorganosiloxane material having a viscosity at 25° C of greater than or equal to 1,000,000 mPa·s. Silicone gums are believed to provide wearability improvement such as long-lasting effect. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. Commercially available silicone gums are described in General Electric Silicone Rubber Product Data Sheets as SE 30, SE 33, SE 54 and SE 76.

The composition of the present invention may further contain a silicone resin, which are highly crosslinked polymeric siloxane systems. Silicone resins are believed to enhance spreadability and improve the feel to the skin. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred are crosslinked silicone powders with tradenames Trefil E-505C, Trefil E-506C, and 9506 Powder; suspensions of silicone elastomer powders with tradenames BY29-119 and BY29-122; and silicone compound emulsions with tradenames SH5500, SC5570, and SM 5571; all available from Dow Corning.

Other useful silicone resins are silicone resin powders such as the material given the CTFA designation polymethylsilsequioxane, which is commercially available as Tospearl™ from Toshiba Silicones.

Silicone materials and silicone resins in particular, can conveniently be identified according to a shorthand nomenclature system well known to those skilled in the art as the “MDTQ” nomenclature. Under this system, the silicone is described according to the presence of various siloxane monomer units which make up the silicone. Briefly, the symbol M denotes the mono-functional unit (CH₃)₃SiO_{0.5}; D denotes the difunctional unit (CH₃)₂SiO; T denotes the trifunctional unit (CH₃)SiO_{1.5}; and Q denotes the quadri- or tetra-functional unit SiO2. Primes of the unit symbols, e.g., M′, D′, T′, and Q′ denote substituents other than methyl, and must be specifically defined for each occurrence. Typical alternate substituents include groups such as vinyl, phenyl, amino, hydroxyl, etc. The molar ratios of the various units, either in terms of subscripts to the symbols indicating the total number of each type of unit in the silicone, or an average thereof, or as specifically indicated ratios in combination with molecular weight, complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000. Commercially available MQ resins are, for example, trimethyl siloxy silicate with tradename BY11-018 available from Dow Corning.

The composition of the present invention may further contain a water-soluble polymer. It is believed that water-soluble polymers provide long-lasting effect. Useful water-soluble polymers include sodium carboxymethyl cellulose, polyvinyl pyrrolidone, polyvinyl alcohol, xanthan gum, agar, pulleran, bentonite, and mixtures thereof. Commercially available water-soluble polymers include the Carbopol series available from B. F. Goodrich Company, and PVP K-30 available from G.A.F. Chemicals.

The composition of the present invention may further contain an oil swelling clay material which functions as a thickener for the composition. Oil swelling clay materials useful herein include hectorite, bentonite, montmorillonite, and bentone clays which have been modified to be compatible with oil. Preferably, the modification is quaternization with an ammonium compound. Preferable oil swelling clay materials include quaternary ammonium modified hectorite. Commercially available oil swelling clay materials include benzyldimethyl stearyl ammonium hectorite with tradename Bentone 38 CG OR available from Rheox. Inc.

The composition of the present invention may further comprise an acryl alkyl silicone copolymer having a melting point of no more than about 40° C. The acryl alkyl silicone copolymers useful herein include those that are graft copolymers of an (meth)acrylic main chain and siloxane side chains, wherein the (meth)acrylic main chain has attached to it an alkyl chain of 16-22 carbons. The acryl alkyl silicone copolymer is believed to provide smoothness to the composition. Commercially available acryl alkyl silicone copolymer include: acrylates/stearyl acrylate/dimethicone methacrylate copolymer with tradename KP-561P available from ShinEtsu Silicone.

Other components which can be formulated into the compositions of the present invention are; emollient mixtures by the tradename MAXI-LIP containing octyl palmitate, tribehenin, sorbitan isostearate and palmitoyl oligopeptide available from Sederma, preservatives such as benzyl alcohol, methyl paraben, propyl paraben, imidazolidinyl area, and EDTA and its salts, perfumes, ultraviolet and infrared screening and absorbing agents such as ethylhexyl methoxycinnamate, and others.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

### Examples 1-5

The following cosmetic compositions are formed by the following components using the method of preparation described herein:

| No. | Component | Ex. 1 | Ex. 2 | Ex. 3 | Ex. 4 | Ex. 5 |
|---|---|---|---|---|---|---|
| 1 | Shea Butter *1 | 5 | 5 | | 3.5 | 5 |
| 2 | Acrylates/Stearyl Acrylate/ | 2 | | 10 | | |
| | Dimethicone Methacrylate Copolymer *2 | | | | | |
| 3 | Emollient mixture of Octyl Palmitate, | 0.5 | 1 | | 1 | |
| | Tribehenin, Sorbitan Isostearate and | | | | | |
| | Palmitoyl Oligopeptide *3 | | | | | |
| 4 | Phenyl Trimethicone *4 | 10 | 15 | | 20 | 10 |
| 5 | Phytosteryl/Octyldodecyl Lauroyl | 12.5 | | | 10 | 5 |
| | Glutamate *5 | | | | | |
| 6 | Phytosteryl Isostearyl Dimer | 15 | 10 | | 15 | |
| | Dilinoleate *6 | | | | | |
| 7 | Macadamia Integrifolia Nut Oil *7 | 5 | | 5 | 5 | 5 |
| 8 | Limnathes Alba (Meadowfoam) Seed Oil *8 | 5 | | | 5 | 5 |
| 9 | Hydrogenated Polyisobutene *9 | | 10 | | 15 | |
| 10 | Tridecyl Isononanoate *10 | | 10 | 4.5 | | |
| 11 | Trimethylsiloxysilicate/ | | | 45 | | |
| | Cyclomethicone *11 | | | | | |
| 12 | Ozokerite Wax *12 | 2 | 2 | 6 | 2 | 1 |
| 13 | Microcrystalline Wax *13 | 2 | 1 | 3 | 2 | 1 |
| 14 | Candelilla Wax *14 | 0.5 | | 4 | 0.5 | |
| 15 | Polyethylene Wax *15 | 5.5 | 7 | 2 | 5.5 | 5 |
| 16 | Polyglyceryl-2 Triisostearate *16 | 7.5 | 10 | | 10 | 7.5 |
| 17 | Polyglyceryl-10 Nonaisostearate *17 | 16.9 | 17.6 | 9 | 9 | 14.8 |
| 18 | Glycerin *18 | 0.09 | 0.7 | 0.7 | 0.18 | 0.09 |
| 19 | Glucosyl Hesperidin *19 | 0.01 | | | 0.1 | 0.02 |
| 20 | Niaciamide *20 | | 0.2 | | | |
| 21 | Urea *21 | | | 0.3 | | |
| 22 | Preservatives | 0.5 | 0.5 | 0.5 | 0.5 | 0.5 |
| 23 | Coloring Powders | 10 | 10 | 10 | | 40 |

Definitions of Components
*1 Shea Butter: CROPURE SB available from Croda
*2 Acrylates/Stearyl Acrylate/Dimethicone Methacrylate Copolymer: KP-561P available from ShinEtsu Silicone
*3 Emollient mixture of Octyl Palmitate, Tribehenin, Sorbitan Isostearate and Palmitoyl Oligopeptide: MAXI-LIP available from Sederma
*4 Phenyl Trimethicone: Silicone Oil KF-56 available from ShinEtsu Silicone
*5 Phytosteryl/Octyldodecyl Lauroyl Glutamate: ELDEW PS-203 available from Ajinomoto Ltd.
*6 Phytosteryl Isostearyl Dimer Dilinoleate: LUSPLAN PI-DA available from Nippon Fine Chemical Co., Ltd.
*7 Macadamia Integrifolia Nut Oil: FLORAMAC HAWAIIAN MACADAMIA available from FLORATECH
*8 Limnathes Alba (Meadowfoam) Seed Oil: CROPURE MDF available from Croda
*9 Hydrogenated Polyisobutene: Parleam-18 available from NOF Corp.
*10 Tridecyl Isononanoate: Tridecyl Isononanoate available from Croda
*11 Trimethylsiloxysilicate/Cyclomethicone: Trimethylsiloxysilicate Cyclomethicone D5 Blend available from Toshiba Silicones.
*12 Ozokerite Wax: Ozokerite Wax SP-1021 available from Strahl & Pitsch
*13 Microcrystalline Wax: MULTIWAX 180-M Yellow available from Witco Chemical
*14 Candelilla Wax: Candelilla Wax NC-1630 available from Cerarica Noda Co., Ltd.
*15 Polyethylene Wax: PERFORMALENE PL available from New Phase Technology
*16 Polyglyceryl-2 Triisostearate: COSMOL 43V available from Nisshin Oil Mills, Ltd.
*17 Polyglyceryl-10 Nonaisostearate: S-FACE IS-1009P available from Sakamoto Pharmaceuticals.
*18 Glycerin: Concentrated Glycerin available from Asahi Denka.
*19 Glucosyl Hesperidin: Glucosyl Hesperidin available from Hayashibara Biochemical Laboratories Inc.
*20 Niaciamide: Niaciamide USP available from Roche Vitamins Inc.
*21 Urea: Urea EP available from Kanto Chemical Co., Ltd.

### Method of Preparation

The make-up compositions of Examples 1-5 are suitably prepared as follows: First, component numbers 18-21 are heated to 50-60° C, and stirred until completely dissolved. Next, a sufficient part of component number 17 is added to the solution, and further stirred until completely dissolved. Separately, component numbers 1 through 16 and remainder of component number 17 are heated to disperse using a Propeller Mixer at 95° C. in a sealed tank. After all components are melted, the mixture is cooled to 85° C. After that, the solution of component numbers 17-21 are added to the tank, and the mixture is dispersed at 85° C. Finally, the dispersion is filled in an air-tight container and allowed to cool to room temperature.

Examples 1 through 3 provide lipsticks, Example 4 provides lipgloss, and Example 5 provides foundation. These embodiments represented by the previous examples have many advantages. For example, the cosmetic compositions provide improved texture of the lips or skin, while also being moisturizing on the lips or skin, and have good physical stability. Examples 1, 2, 4, and 5 further provide brightness of lips and enhanced skin tone by enhancing of blood circulation.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A lipophilic personal care composition comprising by weight:
(a) from about 1% to about 30% of a polyglycerin fatty acid ester having a polyglycerin of 2-20 units and at least one branched fatty acid residue of 8 to 22 carbons;
(b) from about 0.01% to about 6% of glycerin, wherein the amount of glycerin is no more than 20% of the polyglycerin fatty acid ester;
(c) from about 0.001% to no more than an amount dissolvable in glycerin of a water-soluble skin benefit agent; and
(d) a lipophilic carrier which is substantially free of surfactant.

2. The lipophilic personal care composition of claim 1 wherein the water-soluble skin benefit agent is selected from the group consisting of flavonoid compounds, vitamin B3 compounds, ascorbic acid compounds, panthenol, bacterial cultured mediums, allantoin, sodium lactate, PCA soda, amino acids, urea, sodium hyaluronate, chondroitin sulfate, collagen, elastin, pectin, carageenan, sodium alginate, trehalose, tuberose saccharide, chitin derivatives, chitosan derivatives, water-soluble plant extracts, and mixtures thereof.

3. The lipophilic personal care composition of claim 1 wherein the water-soluble skin benefit agent comprises flavonoid compounds.

4. The lipophilic personal care composition of claim 1 wherein the water-soluble skin benefit agent comprises vitamin B3 compounds.

5. The lipophilic personal care composition of claim 1 wherein the lipophilic carrier comprises an N-long chain acyl acidic amino acid ester of sterol having formula (I):
XOC(O)—(CH2)n-CH(NHCOR)—C(O)OY (I)
wherein one of X and Y is an ester residue of sterol selected from the group consisting of phytosterol, cholesterol, lanosterol, stigmasterol, its hydrogenates, and its derivatives, and the other is selected from H, an alkyl or alkenyl of 8-30 carbons, and ester residue of monohydric solid alcohol of 12-38 carbons; COR is a long chain acyl group of 8-22 carbons; and n is an integer of 1 or 2; and having a water holding capability of at least 400%, and is liquid at 25° C.

6. The lipophilic personal care composition of claim 1 wherein the lipophilic carrier comprises a dimer acid ester having a water holding capability of at least 240%, and is liquid at 25° C.

7. The lipophilic personal care composition of claim 1 wherein the composition is prepared by the steps of:
(1) dissolving the water-soluble skin benefit agent into glycerin;
(2) dissolving the product of step (1) into the polyglycerin fatty acid ester; and
(3) mixing the product of step (2) with the anhydrous lipophilic carrier.

8. The lipophilic personal care composition of claim 7 for use as a lipstick, the lipstick comprising by weight:
(a) from about 5% to about 30% of the polyglycerin fatty acid ester;
(b) from about 1% to about 6% of glycerin; and
(c) from about 0.001% to about 5% of the water-soluble skin benefit agent.

9. The lipstick composition of claim 8 further comprising a solid wax.

10. The lipstick composition of claim 8 further comprising a color powder.
